(19) United States
(12) Patent Application Publication
(10) Pub. No.: US 2010/0130442 A1
(43) Pub. Date: May 27, 2010

(54) Lung Injury Treatment

(76) Inventors: Raj Wadgaonkar, Woodmere, NY (US); Richard A. Gross, Plainview, NY (US); Daniel Butnariu, Brooklyn, NY (US); Vipul Patel, Jersey City, NJ (US); Kaumudi Somnay, Woodmere, NY (US)

Correspondence Address: RYAN, MASON & LEWIS, LLP, 90 FOREST AVENUE, LOCUST VALLEY, NY, 11560, US

(21) Appl. No.: 12/598,363

(22) PCT Filed: May 6, 2008
(86) PCT No.: PCT/US08/62759

§ 371 Date: Oct. 30, 2009

Related U.S. Application Data
(60) Provisional application No. 60/916,457, filed on May 7, 2007.

(51) Int. Cl. A61K 31/7016; A61P 11/00; C07H 3/04

(52) U.S. Cl. 514/53; 536/123.13

(57) ABSTRACT

Techniques for lung injury treatment are provided. For example, a technique for treating a lung injury in a patient includes the step of administering a therapeutically effective amount of a sophorolipid to the patient.

### CROSS-REFERENCE TO RELATED APPLICATION(S)

This application claims the benefit of U.S. Provisional Application Ser. No. 60/916,457, filed on May 7, 2007, the disclosure of which is incorporated by reference herein.

### FIELD OF THE INVENTION

The present invention relates generally to immunology and, more particularly, to lung injury treatment.

### BACKGROUND OF THE INVENTION

Acute lung injury (ALI) and acute respiratory distress syndrome (ARDS) are exemplary lung injuries, as well as being devastating diseases with overall mortality rates of 30-40%. ALI and the more severe ARDS represent a spectrum of common syndrome in response to a variety of infectious and non-infectious insults. The syndrome is characterized by flooding of alveolar spaces with a protein-rich exudates, and inflammation that impairs pulmonary gas exchange leading to arterial hypoxemia and respiratory failure. ALI or ARDS may occur in any patient without any predisposition and are triggered mostly by underlying processes such as, for example, acid aspiration, pneumonia, trauma, multiple transfusions, sepsis and pancreatitis. Despite ongoing and intensive scientific research in this area, the mechanisms underlying ALI and ARDS are still not completely understood. Treatment for ALI and ARDS, however, remains largely supportive, without therapies that target specific pathogenetic mechanisms.

Derangements in lung vascular permeability, particularly in the context of ALI, represent a common yet difficult clinical problem associated with increased morbidity and mortality. Effective therapies for the vascular leak associated with ALI are currently not available among existing approaches. Despite recent advances in low tidal volume mechanical ventilation and a better understanding of the underlying pathophysiology of ALI, there remain few effective treatments for this devastating illness among existing approaches.

Vascular endothelial cells, one of the key targets in a lung injury, reside at the plasma/tissue interface. The plasma/tissue interface with endothelial cell lining is distinguished by its versatility and ability to modulate its surroundings to participate in fundamental processes to control clotting, inflammation, and vascular tone. The molecular mechanisms of endothelial apoptosis and necrosis in the initial injury and survival pathways involved in ALI are not well defined. Also, a pharmacological treatment to regulate endothelial activation and severity of vascular injury is not available in existing approaches.

Aspiration induced lung injury (AILI) is the one of the most common and exemplary causes of ARDS. The mortality rate for ARDS resulting from acid aspiration ranges from between 40-50%. Although many supportive therapies have been developed for patients with AILI, no pharmacologic treatment is currently available among existing approaches.

A majority of intensive care patients require mechanical ventilation for life support. Mechanical ventilation is also often used to relieve acute severe respiratory distress. Unfortunately, mechanical stresses effectuated by mechanical ventilation can cause further damage to the lungs and result in further organ failure such as, for example, that of the kidneys. Mechanical ventilation at high tidal volume can induce or enhance lung injury (ventilator induced lung injury (VILI)) leading to a systemic inflammatory response and end-organ dysfunction. “Protective” ventilator strategies were designed in order to prevent significant mortality and morbidity associated with VILI. However, existing approaches including these strategies cannot avoid lung injury induced by, as an example, ventilator in patients with ARDS with heterogeneous injury pattern.

Additionally, existing adjunctive therapies designed to limit the duration of mechanical ventilation such as, for example, surfactant administration or corticosteroid therapy, have not proven beneficial for treating adults with ALI. As a marked increase in vascular permeability with vascular leak into lung tissues is recognized as the central pathogenic cellular mechanism underlying the physiologic derangement characteristic of ALI, novel therapies that reduce lung microvascular permeability are likely to be clinically advantageous.

Accordingly, there exists a need for techniques to more advantageously treat lung injuries.

### SUMMARY OF THE INVENTION

Principles of the present invention provide techniques for treating a lung injury in a patient. For example, in one aspect of the invention, a technique for treating a lung injury in a patient includes the step of administering a therapeutically effective amount of a sophorolipid to the patient.

These and other features and advantages of the present invention will become apparent from the following detailed description of illustrative embodiments thereof, which is to be read in connection with the accompanying drawings.

### BRIEF DESCRIPTION OF THE DRAWINGS

FIG. 1 is a diagram illustrating lung weight and bronchoalveolar lavage (BAL) cell count of a control specimen versus a specimen with lipopolysaccharide-(LPS-) induced lung injury, according to an embodiment of the present invention;

FIG. 2 is a diagram illustrating a magnified image of a control specimen versus a specimen with lipopolysaccharide-(LPS-) induced lung injury, according to an embodiment of the present invention;

FIG. 3 is a diagram illustrating an exemplary depiction of the structure of a sophorolipid, according to an embodiment of the present invention;

FIG. 4 is a diagram illustrating the effect of sophorolipids on LPS-induced lung injury with respect to weight of mice, according to an embodiment of the present invention;

FIG. 5 is a diagram illustrating total lung weight under various conditions, according to an embodiment of the present invention;

FIG. 6 is a diagram illustrating BAL cell count under various conditions, according to an embodiment of the present invention;

FIG. 7 is a diagram illustrating a myeloperoxidase (MPO) assay of bronchoalveolar lavage under various conditions, according to an embodiment of the present invention;

FIG. 8 is a diagram illustrating an MPO assay of lung tissue lysates under various conditions, according to an embodiment of the present invention;

FIG. 9 is a diagram illustrating total protein in lavage under various conditions, according to an embodiment of the present invention;

FIG. 10 is a diagram illustrating total protein in lung lysate under various conditions, according to an embodiment of the present invention;

FIG. 11 is a diagram illustrating a histopathological examination under various conditions, according to an embodiment of the present invention;

FIG. 12 is a diagram illustrating effects of sophorolipids on a specimen with ventilator associated lung injury, according to an embodiment of the present invention; and

FIG. 13 is a diagram illustrating inhibition of acid-induced lung injury by sophorolipids, according to an embodiment of the present invention.

### DETAILED DESCRIPTION OF THE INVENTION

Recent studies have shown that sphinogolipids, specifically Sphingosine-1-Phosphate, attenuates a lung injury induced by intratracheal LPS in spontaneously ventilating C57BL/6 mice. Also, mechanical ventilation induced lung injury was shown to be blocked by Sphingosine-1-Phosphate. Principles of the present invention illustrate that natural molecules like bioactive lipids are effective techniques for attenuating vascular injuries.

Additionally, the term “patient” as used herein is intended to refer broadly to mammalian subjects, and more preferably refers to humans receiving medical attention (for example, diagnosis, monitoring, etc.), care or treatment. Also, a “therapeutically effective amount” of a given compound in a treatment methodology may be defined herein as an amount sufficient to produce a measurable attenuation of a lung injury in the patient.

As described herein, in vitro techniques were developed to examine lung endothelial cell injury and in vivo animal models to understand the mechanisms of lung injury. Using mechanical ventilation, intratracheal instillation of acid, lipopolysaccharides, and bleomycin, lung injury models were developed in mice and rats. Several bioactive lipids with potential surfactant and/or inhibitors of edema properties in mice were tested. Experimental settings mimicked bedside conditions in mice so that the pathological states could be examined in greater detail, and therapeutic treatments could be devised and tested to their relative efficacy.

By way of example only and without limitation, one of the groups of glycolipids was derived from Candida bombicola. This group of glycolipids, known as sophorolipids, was tested further. In a preferred embodiment, sophorolipids are produced by cells of Candida bombicola when grown on carbohydrates, fatty acids, hydrocarbons or their mixtures. Studies using culture supernatants or isolates from the culture broth of sophorolipids have shown to cause reduction in surface tension up to 26 milli-Newtons per meter (mN/m). A sophorolipid has a hydrophilic and a lipophilic part, wherein the hydrophilic portion is a dimeric sugar sophorose, while the lipophilic part is a long chain fatty acid. Up to nine different classes of sophorolipids have been observed that exhibit differences in the length of a fatty acid component.

As illustrated herein, a sophorolipid is a bioactive lipid with surfactant activity that decreases vascular leak associated with, for example, ALI or ARDS. One or more embodiments of the invention attenuate lung injury via inhibition of vascular leak associated with various inflammatory mediators.

Principles of the present invention include administering a therapeutically effective amount of a sophorolipid to a patient with a lung injury. The lung injury may include, for example, acute lung injury (ALI), acute respiratory distress syndrome (ARDS), aspiration induced lung injury (AILI), ventilator induced lung injury (VILI), pulmonary artery ligation, and acid-induced lung injury.

In one or more embodiments of the invention, a sophorolipid may be administered to a patient, for example, intravenously, intramuscularly, as an inhalant, subcutaneously, and/or systemically. A therapeutically effective amount of a sophorolipid may be administered to a patient, for example, one hour after onset of the lung injury and/or six to twenty-four hours after onset of the lung injury. In one or more embodiments of the invention, a sophorolipid may be administered in an amount in the range of 0.1-0.5 milligram per kilogram of body weight (mg/kg). It is to be appreciated, however, that the present invention is not limited to this specific range. For instance, a higher range may be adapted in connection with bigger animals including, for example, dogs, baboons and/or primates. Also, a therapeutically effective amount of a sophorolipid may be administered to a patient one or more times daily for a period of one or more days.

In one or more embodiments of the present invention, a therapeutically effective amount of a sophorolipid is administered to a patient to, for example, attenuate lipopolysaccharides-(LPS-) induced lung injury, decrease bronchoalveolar lavage (BAL) cell count, decrease neutrophil myeloperoxidase (MPO) activity, inhibit vascular leak (in, for example, VILI, LPS-induced lung injury and AILI), and/or attenuate thrombin-induced increases in endothelial monolayer permeability changes.

Sophorolipids are not synthetic inhibitors. Rather, they are bioactive lipids derived from yeast cells (for example yeast cells of Candida bombicola). As illustrated herein, natural bioactive lipids used as pharmacological inhibitors are effective therapy for attenuating vascular injury. Furthermore, as noted above, existing approaches in lung injury treatment do not include or provide these types of inhibitors.

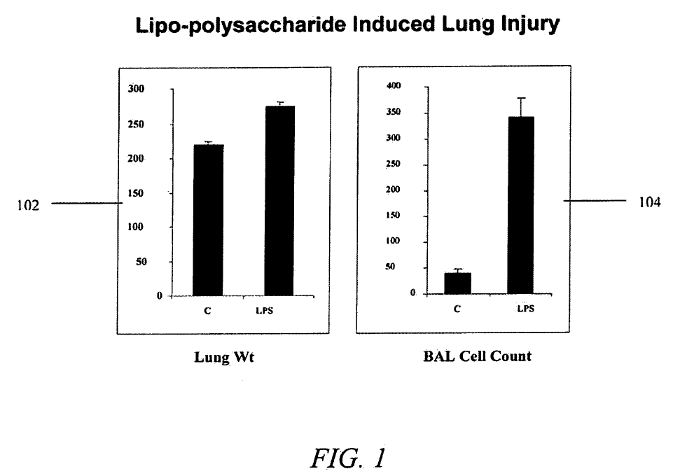

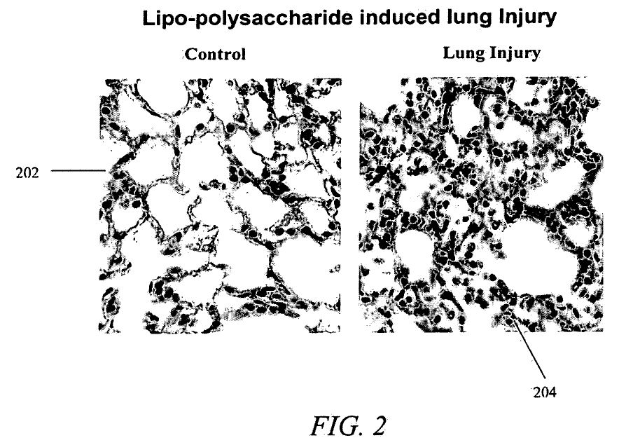

FIG. 1 is a diagram illustrating lung weight 102 and BAL cell count 104 of a control specimen versus a specimen with lipopolysaccharide-(LPS-) induced lung injury, according to an embodiment of the present invention. By way of illustration, FIG. 1 depicts increases in both lung weight and BAL cell count in a specimen with LPS-induced lung injury versus those of a control specimen. Also, FIG. 2 is a diagram illustrating a magnified image of a control specimen 202 versus a specimen with LPS-induced lung injury 204, according to an embodiment of the present invention.

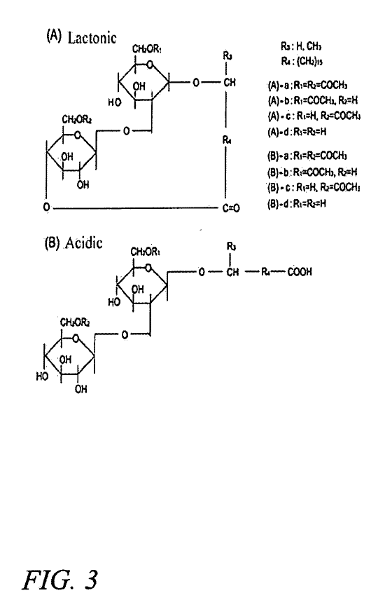

FIG. 3 is a diagram illustrating an exemplary depiction of the structure of a sophorolipid, according to an embodiment of the present invention. The structure of sophorolipid includes a dimeric sugar (sophorose) and a hydroxyl fatty acid, linked by an h-glycosidic bond. There are two types of sophorolipid: acidic sophorolipid and lactonic sophorolipid. Up to nine different structural classes of sophorolipids have been observed.

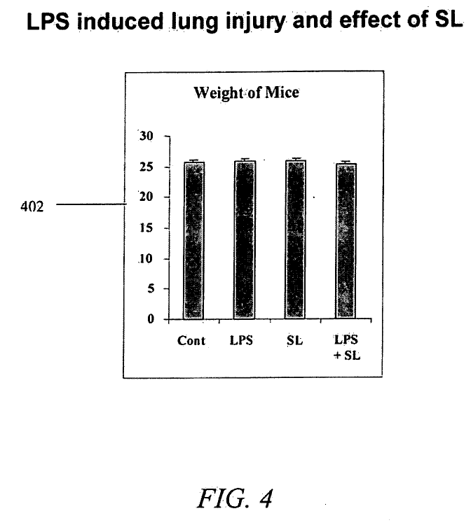

FIG. 4 is a diagram 402 illustrating the effect of sophorolipids on LPS-induced lung injury with respect to weight of mice, according to an embodiment of the present invention. The animals were 8-10 week-old C57BL/6J mice (purchased from the Jackson Laboratory). Intravenous sophorolipid (SL) (0.1 mg/kg) was injected after fifteen minutes, and the mice were divided into four groups: untreated mice (sham surgery and anesthesia), LPS (Sigma-Aldrich, Lot # L 3129), sophorolipid alone, and LPS with sophorolipid.

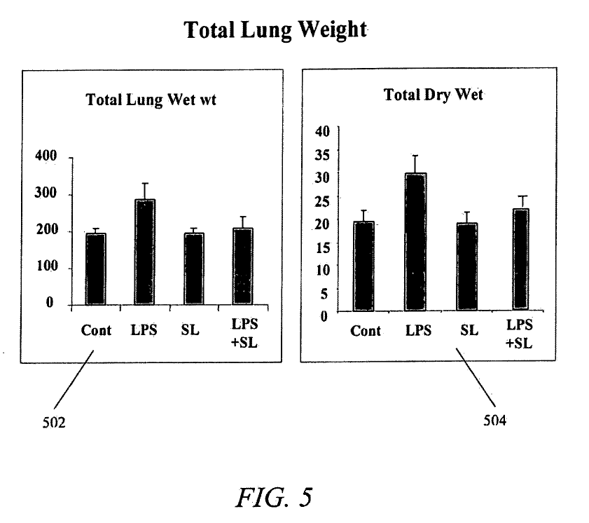

FIG. 5 is a diagram illustrating total lung weight under various conditions, according to an embodiment of the present invention. The figure illustrates a 30% decrease in total lung wet weight in graph 502 and a 27% decrease in total dry weight in graph 504.

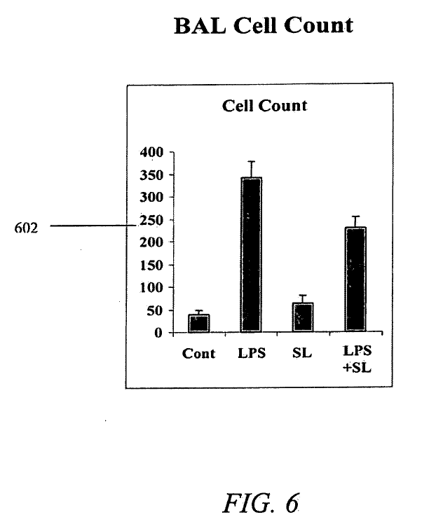

FIG. 6 is a diagram illustrating BAL cell count under various conditions 602, according to an embodiment of the present invention. Lungs were lavaged by 2 milli-liters (ml) aliquots of Hanks' balanced salt solution. Red blood cells in lavage were lysed with ACK lysis buffer and samples were then processed for cell count. Cell counts were done with hemocytometer, and, as illustrated by the figure, there was a resulting 33% decrease in total cell count.

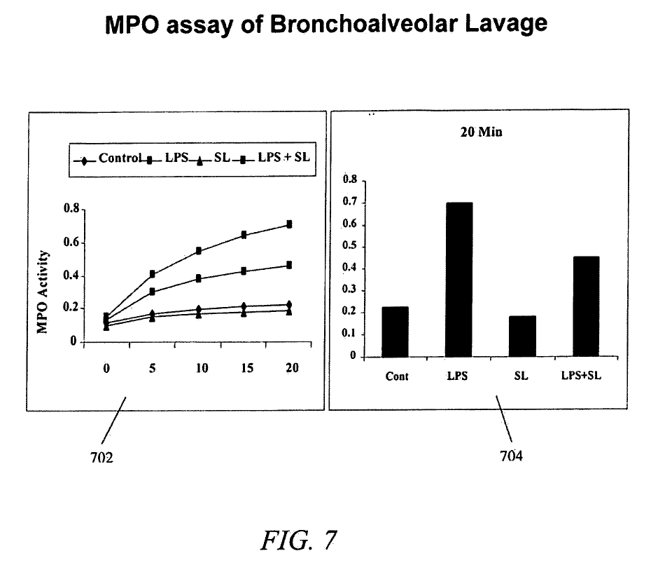

FIG. 7 is a diagram illustrating an MPO assay of bronchoalveolar lavage under various conditions in graphs 702 and 704, according to an embodiment of the present invention. By way of illustration, FIG. 7 depicts increased MPO activity under conditions including LPS and LPS+SL treatment in contrast to conditions including SL treatment and control.

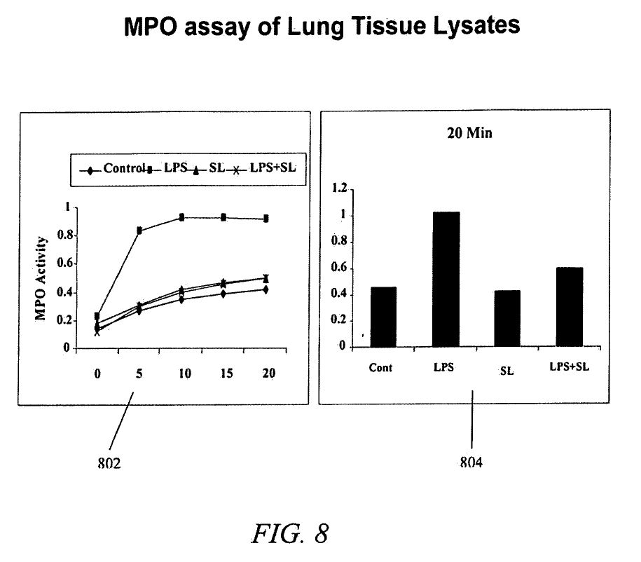

FIG. 8 is a diagram illustrating an MPO assay of lung tissue lysates under various conditions in graphs 802 and 804, according to an embodiment of the present invention. By way of illustration, FIG. 8 depicts increased MPO activity under conditions including LPS and LPS+SL treatment in contrast to conditions including SL treatment and control.

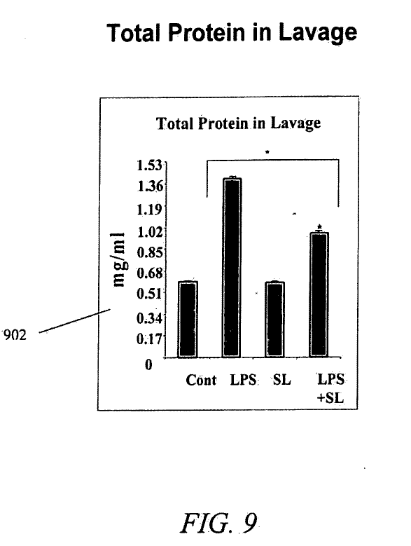

FIG. 9 is a diagram illustrating total protein in lavage under various conditions in graph 902, according to an embodiment of the present invention. Total protein was measured from BAL fluid by standard block save addition (BSA) techniques. The figure depicts a 31% decrease in protein secretion in lavage fluid with sophorolipids.

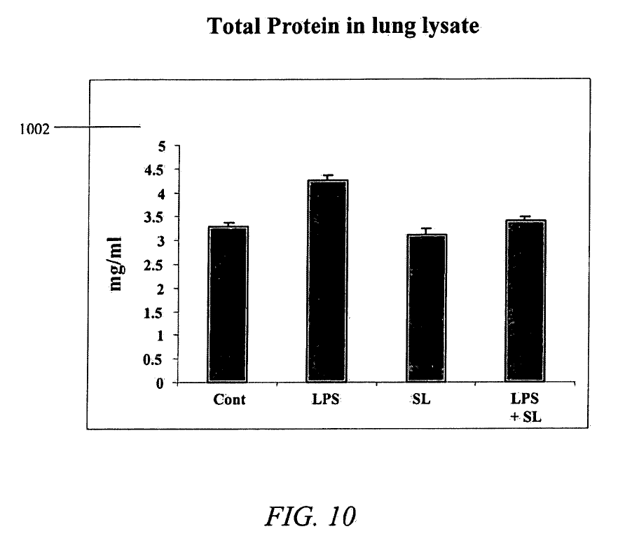

FIG. 10 is a diagram illustrating total protein in lung lysate under various conditions in graph 1002, according to an embodiment of the present invention. By way of illustration, FIG. 10 depicts increased total protein levels under conditions including LPS and LPS+SL treatment in contrast to conditions including SL treatment and control.

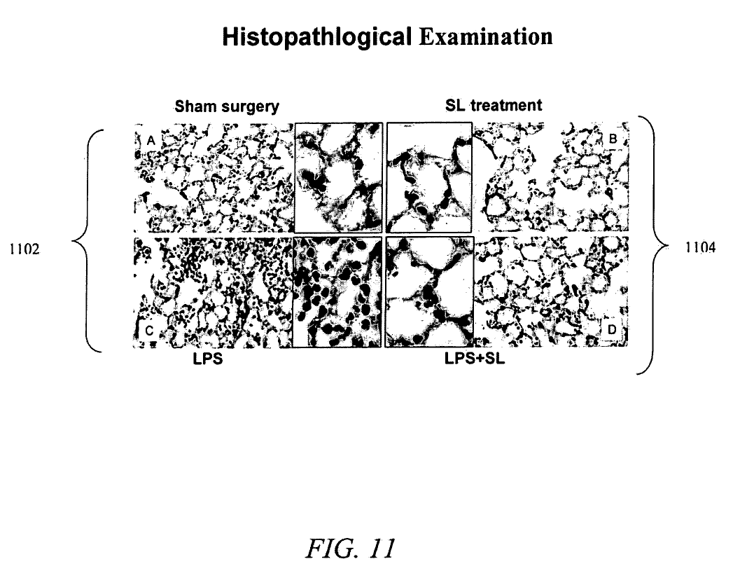

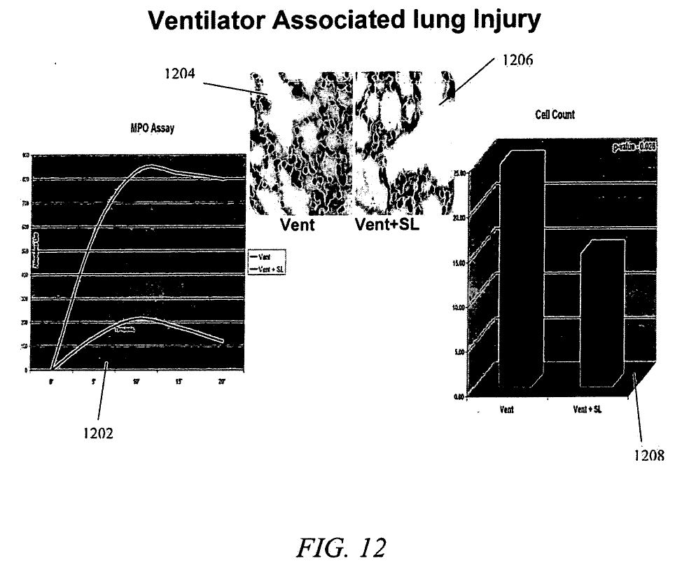

FIG. 11 is a diagram illustrating a histopathological examination under various conditions in images 1102 and 1104, according to an embodiment of the present invention. Also, FIG. 12 is a diagram illustrating effects of sophorolipids on a specimen with ventilator associated lung injury in images 1204 and 1206, according to an embodiment of the present invention. By way of illustration, FIG. 12 depicts increased MPO activity and cell count under conditions of ventilator associated lung injury (Vent) treatment in contrast to conditions including ventilator associated lung injury+SL treatment in graphs 1202 and 1208.

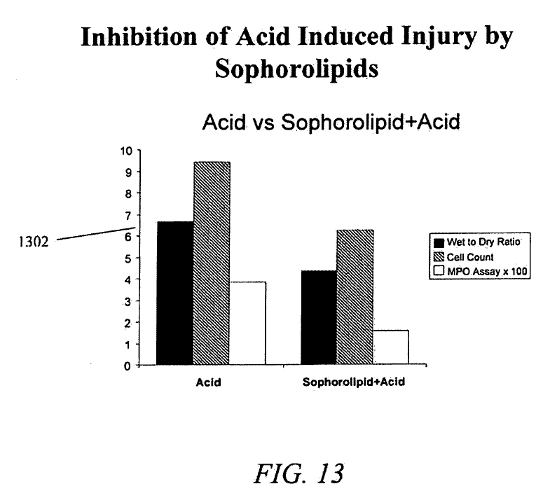

FIG. 13 is a diagram illustrating inhibition of acid-induced lung injury by sophorolipids in graph 1302, according to an embodiment of the present invention. By way of illustration, FIG. 13 depicts decreased wet-to-dry ration, cell count and MPO activity under conditions of sophorolipid and acid-induced injury in contrast to conditions including solely acid-induced injury.

By way of example, one or more embodiments of the invention can be prepared and/or conducted in a manner as described below.

For example, to prepare and treat animals, C57BL/6 mice (8-10 weeks old) are anesthetized with intraperitoneal ketamine (150 mg/kg of body weight) and xylazine 20 mg/kg). The mice are intubated with a 20-gauge (20G) catheter via midline neck incision, lipopolysaccharides (LPS) (2.5 mg/kg) (Lipopolysaccharides from Escherichia coli 0127:B8 -Strain ATCC 12740) or saline (control) is instilled intratracheally. Sophorolipid (0.1 milligram per kilogram (mg/kg)) is injected intravenously 30 minutes after instillation of LPS.

Also, for example, ventilator induced lung injury experimentation can be carried out as follows. C57BL/6 mice (8-10 weeks old) are anesthetized with intraperitoneal ketamine and xylazine. The mice are intubated with a 20G catheter via midline neck incision. The tidal volume used can be 35 milliliter per kilogram (ml/kg). A mixture of sophorolipid (0.1 milligram per kilogram (mg/kg)) is injected intravenously five minutes before starting the ventilation.

Additionally, for example, acid induced lung injury experimentation can be carried out as follows. C57BL/6 mice (8-10 weeks old) are anesthetized with intraperitoneal ketamine and xylazine. The mice are intubated with a 20G catheter via midline neck incision, and hydrochloric acid (HCL) (1 ml/kg) or saline (control) is instilled intratracheally. Sophorolipid (0.1 mg/kg) is injected intravenously 30 minutes after instillation of the acid or saline.

Assessment of a lung injury can include, for example, the following. After 24 hours of observation, the mice are exsanguinated via abdominal aorta transaction. The pulmonary artery of each mouse is cannulated, the left atrial appendage is excised, and 0.5-0.75 ml of phosphate-buffered saline (PBS) is perfused through the pulmonary circulation to remove blood-borne elements. The left lung is then tied off, and the right lung is lavaged by intratracheal injection of three sequential aliquots of Hanks' balanced salt solution. The left lung is then excised en bloc, blotted dry, weighed, and snap-frozen in liquid nitrogen. Measurements are also made, such as, for example, Northern blots, RT-PCR, microarray and proteomics.

A myeloperoxidase activity assay can include, for example, the following. Bronchoalveolar lavage (BAL) and lung lysate myeloperoxidase (MPO) activity, an indicator of neutrophil extravasation, is measured by kinetic readings over 20 minutes with reaction buffer containing potassium phosphate buffer, 0.5% hexadecyltrimethyl ammonium bromide (HTAB), 0.167 mg/ml O-dianisidine dihydrochloride, and 0.0006% hydrogen peroxide (H₂O₂). The rate of change in absorbance is measured at 405 nanometers (nm) on a Vmax kinetic microplate reader with the results adjusted for total lung weight and presented as MPO units/lung.

To characterize the lung morphology, immediately after euthanasia, the left lungs from two animals in each experimental group are inflated to 20 centimeters (cm), and H₂O (water) is used to make 0.2% of low melting agarose for histological examination by hematoxylin and eosin staining.

Performing a BAL fluid cell count can include, for example, the following. The lungs are perfused through the pulmonary circulation to remove the blood-borne elements and plasma as described above. The right lung is tied, and the left lung is lavaged by intratracheal injection of three sequential 0.3 ml aliquots of Hank's balanced salt solution, followed by aspiration. The recovered fluid is pooled and centrifuged. Supernatants were preserved and the leukocyte palette is re-suspended in extraction buffer (50 millimole (mM) potassium phosphate buffer containing 0.5% hexadecyl trimethylammonium bromide-HTAB). Half of this volume is frozen for other analyses, and in the remaining volume red blood cells are lysed with ACK lysing buffer and samples are then processed for cell count with differential. Results are adjusted for total lung volume.

The right lung was removed en bloc and weighed and kept in the incubator for 24 hours, and the dry weight is measured. The wet weight to dry weight ratio is determined and plotted on a graph.

Also, human pulmonary artery endothelial cells (HPAE) are grown to confluence in polycarbonate wells containing evaporated gold microelectrodes in a series with a large gold counter electrode connected to a phase-sensitive lock-in amplifier. Measurements of transendothelial electrical resistance (TER) are performed using an electrical cell-substrate impedance sensing system (ECIS) (Applied BioPhysics Inc., Troy, N.Y., USA). Increases in permeability in an endothelial monolayer are calculated by measuring the changes in resistance of the monolayer.

In connection with the preparatory techniques described above, one or more embodiments of the invention are described below. A lung injury was induced in C57BL/6J mice by high tidal volume ventilation. The tidal volume used was 35 ml/kg. A mixture of sophorolipids was injected intravenously five minutes before starting the ventilation. In one or more embodiments of the present invention, a range of 0.1-0.5 mg/kg of sophorolipids can be used. After six hours of high tidal volume ventilation, the animals were euthanized. Various parameters were used to evaluate the lung injury including, for example, total lung weight, wet to dry ratio, lung tissue myeloperoxidase activity, and BAL fluid cell counts. Lungs were also examined by histopathology.

The lung injury created with high tidal volume ventilation induced a significant increase in wet weight of the lung, cell count of BAL fluid and tissue inflammation in histopathological examination.

After sophorolipid treatment, there was a significant reduction in total lung wet weight (up to 30%), as well as an improved wet to dry weight ratio. Histopathological examination revealed marked reduction in inflammation and neutrophil extravasation in the tissue after sophorolipid treatment. Myeloperoxidase activity, neutrophil count and total protein in BAL were reduced with sophorolipid treatment when compared to mice without treatment.

Sophorolipid treatment significantly attenuated ventilator associated lung injury. In one or more embodiments, sophorolipid treatment attenuated VALI by up to 30%. BAL cell count and neutrophil MPO activity was also decreased, illustrating that sophorolipids inhibit vascular leak.

Compared with the control group, mice treated with sophorolipid before starting ventilation exhibited a significant reduction of wet to dry ratio. In one or more embodiments of the invention, the wet to dry ration was reduced by 21.37% (p-0.017), lung tissue MPO activity was reduced by 74.34% (p-0.033) and BAL fluid cell count was reduced by 40.40% (p-0.026). Significant reduction of inflammatory response was observed by histopathological examination in sophorolipid-treated mice.

Intratracheal instillation of lipopolysaccharide (LPS) in mice is a known model used for assessment of various therapeutic agents in lung injury. C57BL/6J mice were treated with intratracheal LPS (2.5 mg/kg) to induce lung injury. Sophorolipid (0.1 mg/kg) was injected intravenously 30 minutes after instillation of LPS. After 24 hours of observation, the mice were sacrificed and various inflammatory markers were measured including, for example, neutrophil count, myeloperoxidase activity (an indicator of neutrophil extravasation), protein quantity in bronchoalveolar lavage (BAL), and lung tissue myeloperoxidase activity. Also, markers of lung edema such as, for example, total lung weight and wet to dry ratio, were measured. Lungs were also examined by histopathology.

With introduction of LPS intratracheally, marked increases in wet weight of lung, cell count of BAL fluid and tissue inflammation in histopathological examination were observed. In one or more embodiments of the invention, following treatment with sophorolipid in LPS-treated mice there was a reduction in wet as well as dry lung weight by 30%. Inflammatory markers such as, for example, myeloperoxidase activity in BAL, neutrophil count and total protein in BAL were reduced with sophorolipid treatment as compared to mice without treatment. Histopathological examination revealed marked reduction in amounts of inflammation and neutrophil extravasation in tissue in sophorolipid-treated mice.

With respect to acid induced lung injury, 24 male C57BL/6J mice were divided into 4 equal groups: 1) Six mice received intratracheal normal saline solution (NS) alone; 2) Six mice received intravenous injection of sophorolipids and intratracheal NS (Lung injury was induced in 12 C57BL/6J mice via intratracheal instillation of hydrochloric acid (HCl) pH 2.0); 3) Six of these received a mixture of sophorolipids injected intravenously five minutes before instillation of HCl; and 4) The remaining 6 received intracheal HCl.

Four hours after HCl or NS instillation, the animals were euthanized. Various parameters were used to assess lung injury and inflammatory response including, for example, total lung weight, wet to dry ratio, lung tissue myeloperoxidase activity, and BAL fluid cell counts. Lungs were also examined by histopathology.

In one or more embodiments of the present invention, as compared with the control group, mice treated with sophorolipid before AILI showed a significant reduction in wet to dry ratio by 22.3% (p-0.003), lung tissue myeloperoxidase (MPO) activity by 67.5% (p-0.03), and BAL fluid cell counts by 27.53% (p-0.03). Reduction of inflammatory response was also observed by histopathological examination in sophorolipid-treated mice.

Also, one or more embodiments of the invention include mechanisms of sophorolipid induced attenuation of vascular leak (for example, focusing on the role of endothelial cell (EC) activation and barrier dysfunction in lung injury). The EC barrier regulates solute transport between vascular compartments and surrounding tissues functioning as a semi-permeable cellular barrier dynamically regulated by the cytoskeleton. As imbalances in EC barrier function are now characterized by inflammation and increased vascular permeability (including, for example, sepsis, ALI/VALI, and acute respiratory distress syndrome), an understanding of the pathogenic regulatory mechanisms involved has become imperative.

The pleiotropic cytokine, TNF-α, and thrombin lead to increased endothelial permeability in sepsis and related lung injuries. Thrombin, a serine protease, represents an ideal model for the examination of agonist-mediated EC activation and barrier dysfunction, and has been utilized extensively by many laboratories. Thrombin evokes numerous EC responses which regulate hemostasis, thrombosis and vessel wall degenerative pathophysiology, and is recognized as a potentially important mediator in the pathogenesis of ALI. Thrombin is also known to activate the endothelium directly, and to increase albumin permeability across EC monolayers in vitro.

Principles of the present invention illustrate the effect of sophorolipids on thrombin and TNF-induced permeability changes on endothelial monolayer. An endothelial monolayer was first treated with sophorolipids for different time points, and then subjected to agonist such as, for example, thrombin or TNF-α. The effect of sophorolipid treatment on changes in monolayer resistance was measured by TER. The endothelial monolayer incubated with a sophorolipid mixture exhibited a significant decrease in thrombin-induced monolayer gap formation.

In one or more embodiments of the present invention, animal models with acute lung injury have been developed using LPS, acid and ventilator. Sophorolipid treatment significantly attenuated LPS induced lung injury by 30%. Also, BAL cell count and neutrophil MPO activity decreased, illustrating that sophorolipid treatment inhibits vascular leak.

In a preferred embodiment, intravenous administration of sophorolipids significantly reduced the vascular leak in a murine model of ventilator, lipopolysaccharide and acid induced lung injury. Additionally, the effects of thrombin induced increases in endothelial monolayer permeability changes were attenuated by sophorolipid treatment. One or more embodiments of the invention also include, for example, a pharmaceutical composition that includes a therapeutically effective amount of a sophorolipid used to treat a lung injury in a patient.

Although illustrative embodiments of the present invention have been described herein, it is to be understood that the invention is not limited to those precise embodiments, and that various other changes and modifications may be made by one skilled in the art without departing from the scope or spirit of the invention.

What is claimed is:

1. A method of treating a lung injury in a patient, the method comprising the step of administering a therapeutically effective amount of a sophorolipid to the patient.

2. The method of claim 1, wherein the lung injury comprises acute lung injury (ALI).

3. The method of claim 1, wherein the lung injury comprises acute respiratory distress syndrome (ARDS).

4. The method of claim 1, wherein the lung injury comprises aspiration induced lung injury (AILI).

5. The method of claim 1, wherein the lung injury comprises ventilator induced lung injury (VILI).

6. The method of claim 1, wherein the lung injury comprises pulmonary artery ligation.

7. The method of claim 1, wherein the lung injury comprises acid-induced lung injury.

8. The method of claim 1, wherein the sophorolipid is derived from yeast.

9. The method of claim 8, wherein the yeast is Candida bombicola.

10. The method of claim 1, wherein the step of administering a therapeutically effective amount of a sophorolipid to the patient comprises administering the sophorolipid to the patient intravenously.

11. The method of claim 1, wherein the step of administering a therapeutically effective amount of a sophorolipid to the patient comprises administering the sophorolipid to the patient intramuscularly.

12. The method of claim 1, wherein the step of administering a therapeutically effective amount of a sophorolipid to the patient comprises administering the sophorolipid to the patient as an inhalant.

13. The method of claim 1, wherein the step of administering a therapeutically effective amount of a sophorolipid to the patient comprises administering the sophorolipid to the patient subcutaneously.

14. The method of claim 1, wherein the step of administering a therapeutically effective amount of a sophorolipid to the patient comprises administering the sophorolipid to the patient systemically.

15. The method of claim 1, wherein the step of administering a therapeutically effective amount of a sophorolipid to the patient comprises administering the sophorolipid to the patient one hour after onset of the lung injury.

16. The method of claim 1, wherein the step of administering a therapeutically effective amount of a sophorolipid to the patient comprises administering the sophorolipid to the patient six to twenty-four hours after onset of the lung injury.

17. The method of claim 1, wherein the step of administering a therapeutically effective amount of a sophorolipid to the patient comprises administering the sophorolipid in an amount in the range of 0.1-0.5 milligram per kilogram of body weight (mg/kg).

18. The method of claim 1, wherein the step of administering a therapeutically effective amount of a sophorolipid to the patient comprises administering a therapeutically effective amount of the sophorolipid one or more times daily for a period of one or more days.

19. The method of claim 1, wherein the step of administering a therapeutically effective amount of a sophorolipid to the patient comprises administering the sophorolipid to the patient to attenuate lipopolysaccharides-(LPS-) induced lung injury.

20. The method of claim 1, wherein the step of administering a therapeutically effective amount of a sophorolipid to the patient comprises administering the sophorolipid to the patient to decrease bronchoalveolar lavage (BAL) cell count.

21. The method of claim 1, wherein the step of administering a therapeutically effective amount of a sophorolipid to the patient comprises administering the sophorolipid to the patient to decrease neutrophil myeloperoxidase (MPO) activity.

22. The method of claim 1, wherein the step of administering a therapeutically effective amount of a sophorolipid to the patient comprises administering the sophorolipid to the patient to inhibit vascular leak.

23. The method of claim 13, wherein vascular leak is inhibited in at least one of ventilator induced lung injury, lipopolysaccharides-(LPS-) induced lung injury, and acid-induced lung injury.

24. The method of claim 1, wherein the step of administering a therapeutically effective amount of a sophorolipid to the patient comprises administering the sophorolipid to the patient to attenuate thrombin-induced increases in endothelial monolayer permeability changes.

25. A pharmaceutical composition comprising a therapeutically effective amount of a sophorolipid, wherein the pharmaceutical composition treats a lung injury in a patient.
